(19) United States
(12) Patent Application Publication
(10) Pub. No.: US 2003/0114786 A1
(43) Pub. Date: Jun. 19, 2003

(54) Fluid counterbalancing system

(76) Inventors: Joachim Hiller, Goeppingen (DE); Claus Haacke, Melsungen (DE); Rolf Heitmeier, Baunatal (DE)

Correspondence Address: CHRISTIE, PARKER & HALE, LLP, 350 WEST COLORADO BOULEVARD, SUITE 500, PASADENA, CA, 91105, US

(21) Appl. No.: 10/204,338

(22) PCT Filed: Feb. 6, 2001
(86) PCT No.: PCT/EP01/01245

(30) Foreign Application Priority Data
Mar. 9, 2000 (DE) 10011305.8

(51) Int. Cl. A61M 1/00

(52) U.S. Cl. 604/27

(57) ABSTRACT

The quantities of fluid which are fed to a patient (P) by fluid supply devices (10) and the quantities of fluid which are excreted by the patient as urine, wound drainage fluid or similar, received in corresponding receptacles (20a, 20b, 20c) are counterbalanced by a fluid manager (15). The fluid manager (15) controls the fluid supply devices (10) in accordance with the result of the counterbalancing process. Each receptacle (20a, 20b, 20c) comprises a receiving container (22) and a secretion quantity detector (21). The levels are transmitted wirelessly to the fluid manager (15) by data transfer devices (25). Each data transfer device (25) also supplies power to its allocated secretion quantity detector (21) by means of a battery. The data transfer device (25) can be set to a standby state of low power consumption to save battery power. It is awaken by a signal of the fluid manager (15) and set to the transmitting state.

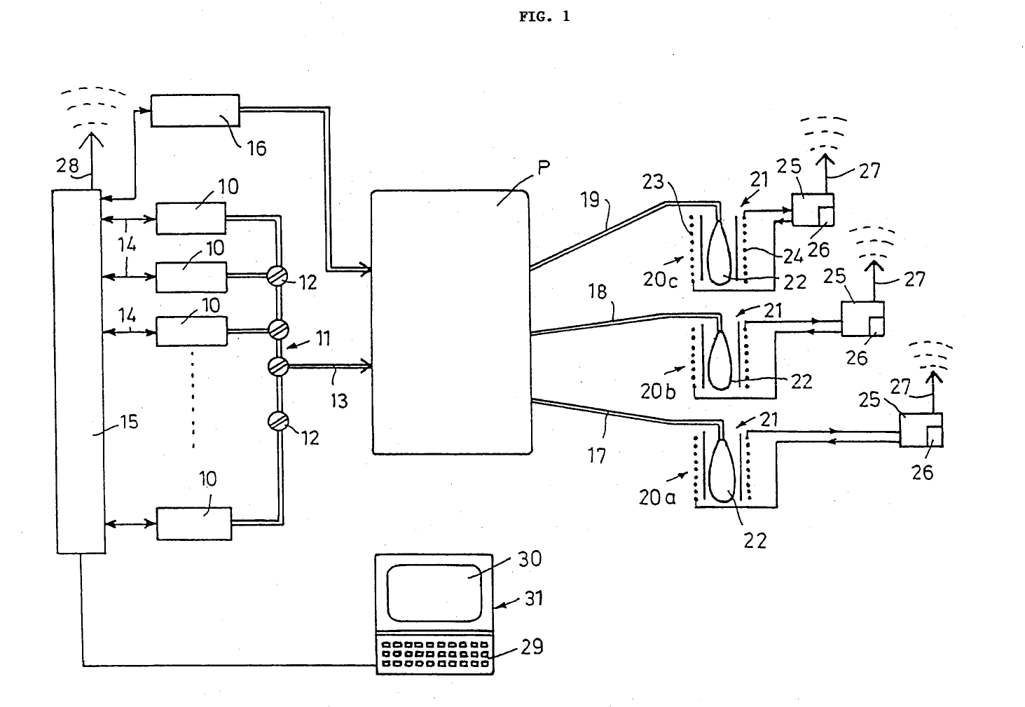

### BACKGROUND OF THE INVENTION

The invention relates to a fluid counterbalancing system for measuring the liquids fed to a patient and the liquids excreted by the patient.

For certain treatments, it is required to measure the liquids fed to the patient by infusion or other administrations and also the liquids and other substances excreted by the patient. The excreted liquids are, e.g., urine, wound drainage liquid and the like. There is a need for a fluid counterbalancing system detecting the sum of all substances fed to the patient and the sum of all substances excreted by the patient to keep the patient's weight at a constant level by controlling it correspondingly. There are counterbalancing systems measuring the patient's weight including the bed to detect whether the patient's weight changes during the medical treatment.

When a patient in need of intensive medical care is connected to numerous infusion apparatus, the numerous electrical cables and fluid lines cause a tangle of lines and the personnel has no adequate overview with sufficient clarity. There is the danger of wrong connections and other grave personnel errors. The operation of a counterbalancing system is extremely personnel-intensive and is susceptible to errors. The personnel has to manually detect the different infusion rates and also measure, add and compare the quantity of the liquids excreted by the patient with the fed liquid quantity. This requires extensive operations of detecting and calculating.

### SUMMARY OF THE INVENTION

It is the object of the present invention to provide a fluid counterbalancing system having a relatively simple and clear structure and being able to automatically draw up a quantitative balance.

This object is solved, according to the invention, with the features indicated in claim 1.

The counterbalancing system according to the invention is provided with a fluid manager controlling the supply rates of the fluid supply devices. The fluid manager is a calculating unit, preferably a microprocessor, forming the central controlling member of the counterbalancing system. The fluid manager is supplied with the signals of the secretion quantity detector provided on the receptacles. In this manner, the fluid manager can detect the sum of the body liquid, i.e. urine and wound secretion, excreted into the receptacles. Depending on this liquid amount, the liquid quantity evaporated through the skin and the liquid quantity excreted otherwise (e.g., by saliva) can be estimated as a percentage. According to given criteria, the amount of the feces excreted by the patient can be estimated as well and considered by the fluid manager. In this manner, the fluid manager draws up a quantitative balance on the basis of the liquid quantities fed to the patient and the liquid quantities excreted by the patient. According to the invention, the secretion quantity detector is provided with a battery-operated mains-independent data transfer device wirelessly communicating with the fluid manager and informing it on the respective level. An essential advantage is that the secretion quantity detector does not need any connection cables, neither a mains cable for the power supply nor a communication cable for the communication with the fluid manager. The receptacle including the secretion quantity detector does not need any external cable connection so that the receptacle in the form of a bag or a rigid collecting system with a suspension device and a level measuring device can be mounted to the patient's bed without any installation of cables being necessary. Both the secretion quantity detector and the data transfer device are provided with battery power so that the entire unit comprising the receptacle, the secretion quantity detector and the data transfer device forms an independently operating unit without a cable connection.

In the transmitting state, the data transfer device has an increased power consumption. Preferably, the data transfer device alternates between a standby state of low power consumption and a transmitting state of higher power consumption. In the standby state, the data transfer device is either switched off or in a sleeping state in which it does not transmit signals but is able to receive signals from the fluid manager. In this manner, the power consumption and the battery load are reduced. When the data transfer device is in the standby state, the secretion quantity detector is in a switched-off or low energy state. It only assumes the measuring state when the data transfer device assumes the transmitting state. In this manner, the power consumption of the secretion quantity detector is also minimized by only activating it when the data transfer device is in the transmitting state.

Preferably, the change between standby state and transmitting state of the data transfer device is controlled by wake-up signals of the fluid manager. In doing this, the fluid manager polls the data transfer devices of the individual fluid receptacles with respect to criteria predetermined by the fluid manager. In this manner, a time selection can be achieved as well since the fluid manager decides which of the plural data transfer devices is requested to transmit at a certain time.

Preferably, the fluid manager is arranged in physical proximity to the fluid supply devices (infusion pumps) to which it is connected. The fluid manager controls the fluid supply devices considering the measured values received by the secretion quantity detectors of the different receiving containers. The fluid manager is provided with an input/output device into which the user can input different parameters and at which he can call a display of the operational states.

Preferably, the secretion quantity detectors are configured as level measuring devices and have the same structure. Receiving containers in the form of bags or rigid containers are inserted therein. An indication about the respective container type is input into the fluid manager. Then, the fluid manager containing the level values supplied by the respective secretion quantity detector makes a quantitative conversion to convert the level values to quantitative values, considering the container form. The fluid manager can receive the indication about the container type from a sensor provided at the respective receptacle and excited by the suspended bag in such a manner that it supplies a code corresponding to the bag type, which is transmitted to the fluid manager by the data transfer device.

Hereinafter, an embodiment of the invention is explained in detail with respect to the only figure of the drawing.

### BRIEF DESCRIPTION OF THE DRAWINGS

In the drawing, a fluid counterbalancing system according to the invention is schematically illustrated.

### DETAILED DESCRIPTION OF THE INVENTION

P denotes a patient lying in a patient's bed and being connected to several fluid supply devices 10 via a tube system. The fluid supply devices 10 are infusion pumps, for example, each of which supplies liquid taken from a syringe, a bag or another container at a set supply rate. Here, the fluid supply devices are connected with a tap bank 11 comprising several operable directional valves 12. Via a catheter 13, the outlet of the tap bank 11 is connected to the body of the patient P.

Via electrical control lines 14, the fluid supply devices 10 are connected to a fluid manager 15 individually controlling the fluid supply devices, particularly the supply rates. The fluid manager 15 is a data processing device adapted to bidirectionally communicate with each fluid supply device.

In this example, the fluid supply devices 10 are infusion apparatus by means of which physiological solutions and medicines can be introduced into the blood system of the patient P. Further, a fluid supply device 16 for the parenteral nourishment of the patient is connected to the fluid manager 15.

Different receptacles are each connected by respective tubes 17, 18, 19 to the body of the patient. The receptacle 20a is for the reception of urine and the receptacles 20b and 20c are each for draining the wound and for receiving the wound secretion.

Each receptacle comprises a secretion quantity detector in the form of a level measuring device 21 and a receiving container 22, here in the form of a flexible bag, inserted in the level measuring device. The receiving container 22 is inserted into the level measuring device 21 in a defined manner so that the latter can detect the liquid level in the receiving container.

The level measuring device is of the radiation barrier type. It includes an array of radiation transmitters 23, preferably LEDs, and an array of radiation receivers 24, preferably phototransistors, on the opposite side. There are different possibilities to detect the level. It can be effected by detecting the attenuation of the emitted light by the liquid and thereby detecting the level. To this end, radiation in the infrared range is particularly suitable. Another possibility is to obliquely emit light radiation onto the bag surface and to make use of the fact that light is diffracted into another direction in the presence of liquid than in the absence of liquid. Such a level measuring device, for example, is described in U.S. Pat. No. 4,745,929.

Each receptacle 20a, 20b, 20c has a battery-operated mains-independent data transfer device 25 containing a battery 26 allocated thereto. The data transfer device 25 provides the level measuring device 21 with electrical power and receives corresponding level signals therefrom, which indicate the height of the respective level in the receiving container 22. Each data transfer device is equipped with an antenna 27 via which it is able to wirelessly communicate with an antenna 28 of the fluid manager 15. The data transfer device 25 can assume a standby state of low power consumption. In the course of this, the level measuring device 21 is in a switched-off or low energy state. Further, the data transfer device can assume a transmitting state. In this transmitting state, the level measuring device 21 is in a measuring state. The control between standby state and transmitting state is effected by signals from the fluid manager 15. The latter determines at which time a data transfer device 25 is selectively put into the transmitting state. Thus, the fluid manager 15 time-selectively calls the data from the respective data transfer devices.

In this manner, level data are respectively transferred to the fluid manager 15 by the data transfer devices. The fluid manager 15 receives information about the type of the receiving container 22 inserted in the respective level measuring device 21. Presently, different suppliers offer bags in different forms and sizes. The characteristic values of these bag types are stored in the fluid manager so that the filling amount in the bag can be calculated on the basis of the level. This quantitative conversion is effected in the fluid manager 15 to which only the respective filling heights are communicated by the data transfer devices.

Based on the body liquids excreted by the patient, the fluid manager 15 calculates the total drain of the patient. The amount of liquid fed to the patient is also determined on the basis of the supply rates of the fluid supply devices 10. In this manner, a change of the patient's weight can be detected very precisely. The fluid manager is connected to an input/output device 31 comprising a keyboard 29 and a screen as display device 30. Via the input/output device 31, the user can communicate certain information to and input data into the fluid manager 15. At the display device 30, he can call the present setting of the fluid supply devices as well as the liquid quantities contained in the receptacles 20a, 20b, 20c. Moreover, the user can change the duration of the standby states of the data transfer devices 25. If certain diuretic medicines are administered, for example, it is required to get a quick reaction of the quantitative balance. In this case, the transmitting states are established in relatively quick succession. During normal operation, however, it is sufficient to switch on the transmitting states about every three minutes.

Instead of a level measuring device 21, another kind of secretion quantity detector can be used as well, a weighing device, for example.

What is claimed is:

1. A fluid counterbalancing system comprising at least one controllable fluid supply device (10) for supplying liquid to a patient (P), at least one receptacle (20a, 20b, 20c) for receiving body secretion of the patient, the receptacle including a receiving container (22) and a secretion quantity detector (21), and a fluid manager (15) controlling the fluid supply device (10), the fluid manager being operatable to establish a quantitative balance of the patient on the basis of the fluid quantities fed by the fluid supply devices (10) and the fluid quantity drained into the receiving container, wherein the secretion quantity detector (21) is provided with a battery-operated mains-independent data transfer device (25) wirelessly communicating with the fluid manager (15) and informing it on the respective measured value detected by the secretion quantity detector.

2. The fluid counterbalancing system according to claim 1, wherein the data transfer device (25) alternately assumes a standby state of low power consumption and a transmitting state of higher power consumption.

3. The fluid counterbalancing system according to claim 1 or 2, wherein the data transfer device (25) transmits level signals as measured values which are converted to volume values in the fluid manager (15).

4. The fluid counterbalancing system according to one of claims 1-3, wherein the change between standby state and transmitting state is controlled by wake-up signals of the fluid manager (15).

5. The fluid counterbalancing system according to one of claims 1-4, wherein in the case of plural secretion quantity detectors (21) each secretion quantity detector reacts to an individual identification signal of the fluid manager (15) and the fluid manager successively transmits the identification signals.

6. The fluid counterbalancing system according to one of claims 1-5, wherein the secretion quantity detectors (21) are of the same structure and different types of receiving containers (22) are provided, an input concerning the respective container type is adapted to be input into the fluid manager (15), and the fluid manager (15) performs a conversion of the measured values supplied by the respective secretion quantity detector (21).

7. The fluid counterbalancing system according to claim 6, wherein the indication about the container type is transferred to the fluid manager (15) by the data transfer device (25).

8. The fluid counterbalancing system according to one of claims 1-7, wherein the fluid manager controls at least one fluid supply device (10) in dependence on the detected liquid amount excreted by the patient.

9. The fluid counterbalancing system according to one of claims 1-8, wherein the secretion quantity detector is a weighing device.

10. The fluid counterbalancing system according to one of claims 1-8, wherein the secretion quantity detector is a level measuring device (21) of the radiation barrier type and comprises an array of radiation transmitters (23) and an array of radiation receivers (24) on the opposite side.

11. The fluid counterbalancing system according to claim 2, wherein the secretion quantity detector (21) assumes a measuring state when the data transfer device (25) assumes the transmitting state, and assumes a switched-off or low energy state when the data transfer device (25) assumes the standby state.
